(19) United States
(12) Patent Application Publication
(10) Pub. No.: US 2009/0216115 A1
(43) Pub. Date: Aug. 27, 2009

(54) Anchoring wirless markers within a human body

(75) Inventors: Keith D. Seiler, Issaquah, WA (US); Eric Hadford, Snohomish, WA (US); Lynn M. Purdy, Sammamish, WA (US)

Correspondence Address: CALYPSO MEDICAL / PERKINS COIE, LLP, P.O. BOX 1247, SEATTLE, WA, 98111-1247, US

(73) Assignee: Calypso Medical Technologies, Inc., Seattle, WA (US)

(21) Appl. No.: 11/658,333

(22) PCT Filed: Jul. 25, 2005
(86) PCT No.: PCT/US05/26430

§ 371 Date: Dec. 1, 2008

Related U.S. Application Data
(60) Provisional application No. 60/590,452, filed on Jul. 23, 2004.

(51) Int. Cl. A61B 5/05

(52) U.S. Cl. 600/426

(57) ABSTRACT

Apparatus and methods for anchoring implanted wireless markers in a patient's body to accurately locate a small target within a soft tissue region. One embodiment of the invention comprises a casing, a transponder partially encased in the casing, and an anchor protruding from the casing. The anchor can either be an extension of the casing or a separate component partly embedded in the casing. Different embodiments of the invention may be well suited for percutaneous implantation and/or surgical implantation.

### CROSS-REFERENCE TO RELATED APPLICATION

This application claims the benefit of and priority to U.S. Provisional Patent Application No. 60/590,452 entitled, “ANCHORING WIRELESS MARKERS WITHIN A HUMAN BODY,” filed Jul. 23, 2004, and which is incorporated in its entirety herein by reference.

### TECHNICAL FIELD

The present invention is directed toward implantable markers with signal transmitters that wirelessly transmit location signals from within a patient's body. In particular, several aspects of the invention relate to anchoring or fastening markers to their surrounding medium to prevent them from changing location.

### BACKGROUND

Many medical procedures require monitoring or treating an internal tissue mass or other parts within a human body. In such applications, medical procedures must accurately locate a small target location within a soft tissue region, an organ, a bone structure, or another body part (e.g., colon, vascular system, etc.). The small target location can be a lesion, polyp, tumor, or another area of interest for monitoring or treatment. For example, it is particularly important to know or estimate the precise location of the target in radiation oncology because it is desirable to accurately determine the accumulated dosage applied to the target and it is detrimental to expose adjacent body parts to the radiation. In applications for treating prostate cancer, for example, it is detrimental to irradiate the colon, bladder or other neighboring body parts with the high-intensity radiation beam. Surgical applications, such as breast surgery and other procedures involving soft tissue, also require knowing the precise location of a target because a lesion in soft tissue is not necessarily fixed relative to external landmarks on the patient.

Some applications are particularly challenging because physicians often need to treat small, non-palpable lesions that cannot be observed. This problem is compounded in soft tissue applications because the soft tissue is mobile and can move with respect to a reference point on the patient. In the case of breast cancer, for example, the location of a non-palpable lesion in the breast is identified at a pre-operative stage using an imaging system. The surgical procedure or radiation treatment, however, occurs at a subsequent point in time, and the patient, and consequently the tissue and the lesion, are typically in a different position during such processes compared to the pre-operative imaging stage. The physician, therefore, generally estimates the location of lesion during the process.

One problem with treating non-palpable lesions in soft tissues is that the physicians may incorrectly estimate the location of the target. As a result, the physician may not remove the entire lesion or cause undesirable collateral damage to healthy tissue by removing a significant amount of tissue proximate to the lesion. The same kind of problems may occur in case of radiation. In general, during the radiation or surgical procedure it is desirable, and in many cases it is vital, to know the precise location of the targets.

In medical fields, to accurately target portions of a human body, various devices are used. For example, different imaging systems have been used to locate areas or particular targets in a patient before performing radiation oncology or surgical procedures. In many medical applications, however, imaging techniques by themselves are not well suited for accurately identifying the actual location of a target. And although x-ray, Magnetic Resonance Imaging (MRI), CT and other imaging techniques are useful to locate targets within the body at a pre-operative stage of a procedure, they are often not suitable or difficult to use in real time during surgery or radiation therapy.

Another technique to locate a target in a patient is to implant a marker relative to the target. For example, implantable markers that generate a signal have been proposed for use to locate a selected target in a patient in radiation oncology procedures. U.S. Pat. No. 6,385,482 B1 issued to Boksberger et al. discloses a device having an implanted emitter unit located inside or as close as possible to a target object, and a plurality of receiver units that are located outside of the patient. The wired device disclosed in Boksberger, however, may not be suitable for use in radiation oncology and many surgical procedures because it is impractical to leave a wired marker implanted in a patient for the period of time of such procedures (e.g., five to forty days).

Another example is the U.S. Pat. No. 5,397,329 to Allen, which describes fiducial implants for a human body that can be detected in x-rays. The fiducial implants are implanted into the skull or other bone structure beneath the skin. The fiducial implants in Allen are also spaced sufficiently far apart from one another to define a plane that can be identified by the imaging system and is used in connection with creation of images of a body portion of interest. The systems disclosed in Allen generally function effectively only when the devices defining the body portion of interest are fixed structures, such as bone, and thus they are not well suited to operate as intended when the devices are inserted in amorphous, pliable tissue.

Another recent method for locating a target within the body includes a wireless implantable marker configured to be implanted, surgically or percutaneously, into a human body relative to a target location. The markers include a casing and a signal element in the casing that wirelessly transmits location signals in response to an excitation energy. One concern of using implanted markers in soft tissues is that the markers may move within the patient after implantation. To resolve this concern, Calypso Medical Technologies, Inc. previously developed several anchors and fasteners for securing the markers to soft tissue structures, as disclosed in U.S. application Ser. No. 10/438,550, which is incorporated herein by reference. Although these anchors work well for percutaneous implantation, they may be improved for surgical applications. Therefore, it would be desirable to further develop markers for surgical and/or percutaneous implantation.

### BRIEF DESCRIPTION OF THE DRAWINGS

FIG. 1 is an isometric view of a marker in accordance with an embodiment of the invention.

FIG. 2 is an isometric view of a marker in accordance with an embodiment of the invention.

FIG. 3 is an isometric view of a marker in accordance with an embodiment of the invention.

FIG. 4 is an isometric view of a marker in accordance with an embodiment of the invention.

FIG. 5 is an isometric view of a marker in accordance with an embodiment of the invention.

FIG. 6 is an isometric view of a marker in accordance with an embodiment of the invention.

FIG. 7 is an isometric view of a marker in accordance with an embodiment of the invention.

FIG. 8 is an isometric view of a marker in accordance with an embodiment of the invention.

FIG. 9 is an isometric view of a marker in accordance with an embodiment of the invention.

FIG. 10 is an isometric view of a marker in accordance with an embodiment of the invention.

FIG. 11 is an isometric view of a marker in accordance with an embodiment of the invention.

FIG. 12 is a side elevation view of a marker in accordance with an embodiment of the invention.

FIG. 13 is an isometric view of a marker in accordance with an embodiment of the invention.

FIG. 14 is an isometric view of a marker anchoring system in accordance with an embodiment of the invention.

FIG. 15 is a side elevation view of a marker anchoring system in accordance with an embodiment of the invention.

FIG. 16 is an isometric view of a marker anchoring system shown implanted in tissue in accordance with an embodiment of the invention.

FIG. 17A is a top plan view of a marker anchoring system in accordance with an embodiment of the invention.

FIG. 17B is a side elevation view of the marker anchoring system of FIG. 17A.

FIG. 18A is a side elevation view of a marker anchoring system in accordance with an embodiment of the invention.

FIG. 18B is a cross-sectional view of a marker anchoring system taken substantially along line 18B-18B of FIG. 18A.

FIG. 19 is a side elevation view of a marker anchoring system in accordance with an embodiment of the invention.

FIG. 20 is an isometric view of a marker anchoring system in accordance with another embodiment.

### DETAILED DESCRIPTION

### A. Overview

The following disclosure describes several embodiments of wireless markers configured to be implanted and anchored within a human in a manner that prevents the markers from migrating from the implantation site. The markers are highly suitable for use in surgical radiation therapy and other applications to determine the location and orientation of a target of the patient. The markers can be anchored or fastened to tissue or another anatomical medium in a number of invasive and non-invasive ways for surgical or percutaneous implantation.

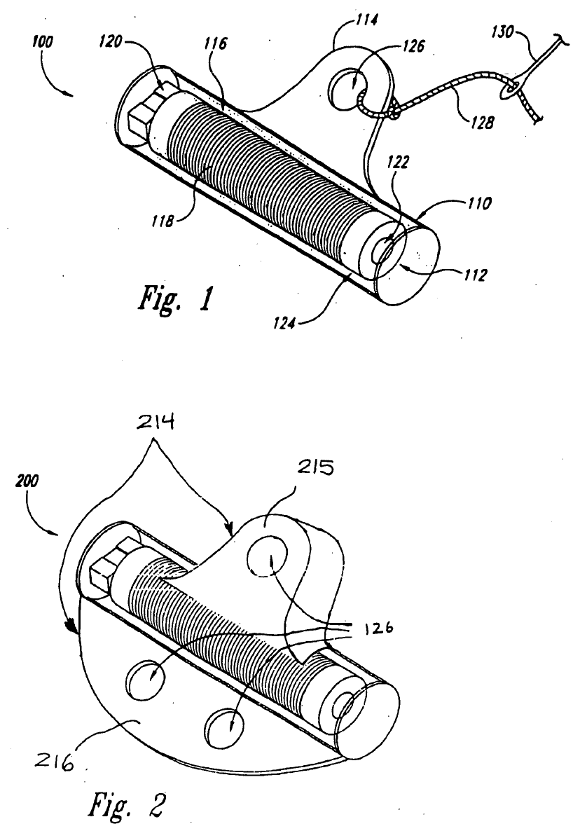

Several embodiments and features of markers with anchors in accordance with embodiments of the invention are set forth and described in FIGS. 1-20. In other embodiments of the invention, the markers can include additional or different features than those shown in FIGS. 1-20. Additionally, several embodiments of markers in accordance with the invention may not include all the features shown in these Figures. For the purposes of brevity, like reference numbers refer to similar or identical components of the markers in FIGS. 1-20.

One embodiment of a marker for localizing a target of a patient comprises a casing, a magnetic transponder at least partially received in the casing, and an anchor carried by the casing. The casing is a biocompatible barrier configured to be implanted in the patient. The casing can be a generally cylindrical capsule that is sized to fit within a needle for percutaneous implantation, but the casing can have other geometric shapes, sizes, and configurations in other applications. For example, the casing can be larger for surgical applications. The magnetic transponder produces a wirelessly transmitted magnetic field in response to a wirelessly transmitted excitation energy. The magnetic transponder can further comprise a magnetic core, a coil wrapped around the core, and a capacitor coupled to the coil. The anchor, which can project from the casing, secures the marker to an anatomical structure to prevent the marker from moving from the implantation site. In one embodiment, the anchor may be sutured to the anatomical structure or attached to the anatomical structure by mechanical members or chemical attributes.

In one embodiment, the anchor protrudes from the casing and has a hole through which a needle, suture line, or other suture material can pass. The hole defines a suture retainer in the protrusion. The anchor of this embodiment may be an integral extension of the casing, or the anchor can be a separate extension embedded in or attached to the casing. The embedded part of an anchor may be formed such that it creates a strong footing in the casing material to better support the protruding portion of the anchor. The anchor, for example, can be a fin or flange with one or more holes for receiving the suture material. In another embodiment, a plurality of anchors may protrude from a casing.

In an alternative embodiment, the marker can have an anchor including a protrusion from the casing, and the suture retainer can be an element configured to retain the suture material without being a completely enclosed hole. For example, open loops, hooks or T-shaped members can be also suitable for suturing a marker to its surrounding tissue.

In additional embodiments of the invention, an anchorable marker for localizing a target of a patient comprises a casing, a transponder that produces a wirelessly transmitted magnetic field in response to a wirelessly transmitted excitation field, and an anchor partially embedded within the casing. The anchor can have a shape and/or material that pierces, engages or otherwise interfaces with the anatomical anchoring site such that the marker cannot be easily dislodged. Such embodiments are also well suited for surgical or percutaneous implantation.

The invention further includes methods for manufacturing and using markers with anchors. One embodiment of such a method comprises providing a transponder that produces a wirelessly transmitted magnetic field in response to a wirelessly transmitted excitation field and forming a casing around the transponder. This method can further include embedding, attaching or forming a suturable anchor in the casing. The suturable anchor can have suturing material, such as a needle and suture wire pre-attached to the anchor. In one embodiment, for example, the needle can be threaded through the anchor and the suture line can be pre-tied to the anchor so that the surgeon can immediately suture the marker to the tissue upon opening of the package. Other combinations of a suturable marker and suturing material are possible with other embodiments of the invention.

In the following description, several specific details are presented to provide a thorough understanding of the embodiments of the invention. One skilled in the relevant art, however, will recognize that the invention can be practiced without one or more of the specific details, or the invention can be practiced in combination with or without other components. Furthermore, the particular features, structures, implementation, or characteristics may be combined in any suitable manner in one or more embodiments. In other instances, well-known implementations or operations are not shown or described in detail to avoid obscuring aspects of various embodiments of the invention.

### B. Embodiments of Anchorable Markers

FIGS. 1-20 are isometric views of markers 100-2000 in accordance with different embodiments of the invention. Referring to FIG. 1, a marker 100 includes a casing 110, a magnetic transponder 112 (e.g., a resonating circuit) at least partially encased in the casing 110, and an anchor 114. The casing 110 is a biocompatible barrier, which can be made from plastics, ceramics, glass or other suitable materials, and the casing 110 is configured to be implanted in the patient. The casing 110 can be a generally cylindrical capsule that is sized to fit within a needle for percutaneous implantation. For example, the casing 110 can have a diameter of approximately 2 mm or less. In surgical applications, the casing can have a larger diameter and other configurations.

The magnetic transponder 112 can include a resonating circuit that produces a wirelessly transmitted signal in response to a wirelessly transmitted excitation field. In one embodiment, the magnetic transponder 112 comprises a coil 116 defined by a plurality of windings of a conductor 118. Many embodiments of the magnetic transponder 112 also include a capacitor 120 coupled to the coil 116. The coil 116 can resonate at a resonant frequency solely using the parasitic capacitance of the windings without having a capacitor, or the resonant frequency can be produced using the combination of the coil 116 and the capacitor 120. The coil 116 accordingly defines a signal transmitter that generates an alternating magnetic field at the selected resonant frequency in response to the excitation energy either by itself or in combination with the capacitor 120. The coil 116 generally has 800-2000 turns, and the windings are preferably wound in a tightly layered coil.

The magnetic transponder 112 can further include a core 122 composed of a material having a suitable magnetic permeability. For example, the core 122 can be a ferromagnetic element composed of ferrite or another material. Suitable embodiments of magnetic transponders are disclosed in U.S. patent application Ser. Nos. 10/334,698 and 10/746,888, which are incorporated herein by reference in their entirety. The magnetic transponder 112 can be secured to the casing 110 by an adhesive 124.

The embodiment of the anchor 114 protrudes from the marker casing 110. The anchor 114 can be an integral extension of the casing 110, or the anchor 114 can be a separate component attached to and/or embedded in the casing 110. When the anchor 114 is a separate component, it can be made from a suitable biocompatible material, such as metals, metal alloys, or polymers and other synthetic materials. An example of one such material is spring steel, although other “memory” metal alloys may be suitable. The anchor 114 shown in FIG. 1 is a fin or flange having a suture retaining element 126 configured to hold a suture line. In the case of a flange, the anchor 114 can be as thick as the diameter of the casing 110. In this embodiment, the suture retainer 126 is an enclosed hole, but in other embodiments the suture retainer 126 does not need to be completely enclosed. A suturing material, such as suture line 128 and needle 130, is pre-attached to the anchor 114 in the embodiment shown in FIG. 1.

FIG. 2 is an isometric view of a marker 200 in accordance with another embodiment of the invention. The marker 200 differs from the marker 100 in that the marker 200 includes an anchor 214 having a first anchor member 215, a second anchor member 216, and a plurality of suture retainers 126. The first anchor member 215 can be a fin or flange similar to the anchor 114 described above. The second anchor member 216 can be a fin or flange extending along a substantial portion of the length of the marker, and the second anchor member 216 has a plurality of suture retainers 126. This embodiment allows a surgeon to secure the marker from different directions to multiple points, and it provides more surface contact area to restrict movement of the marker.

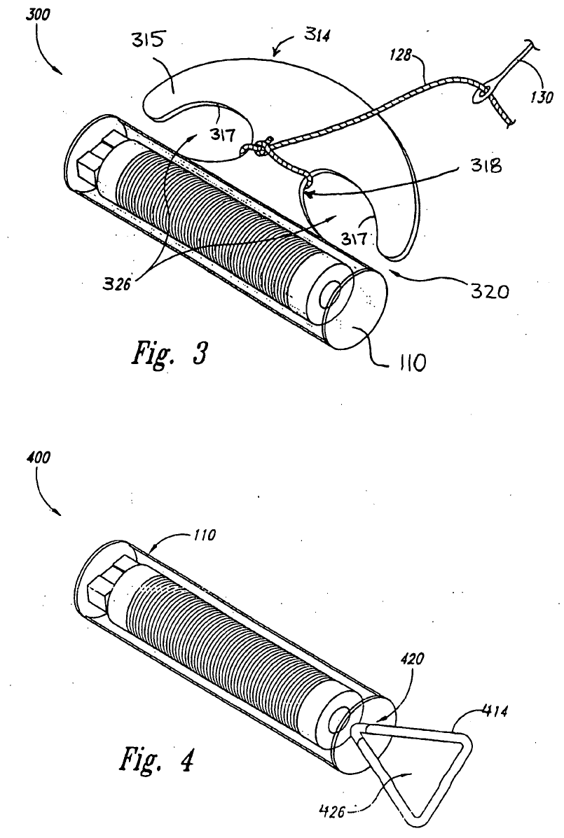

FIG. 3 is an isometric view of a marker 300 in accordance with still another embodiment of the invention. The marker 300 has an anchor 314 with a suture retainer 326 that is not a completely enclosed hole. The anchor 314, more specifically, can be a T-shaped fin projecting from the casing 110, and the anchor 314 can have a first arm 315 extending in one direction and a second arm 316 extending in another direction. The fin is generally relatively thin, but it can have a thickness approximately equal to the diameter of the casing in some embodiments. The first and second arms 315 and 316 can have inner edges 317 that form a partial enclosure. The suture retainer 326 shown in FIG. 3 is defined by the arms 315 and 316. This embodiment allows the suture line 128 to be wrapped around a stem 318 of the anchor 314. The suture line 128, for example, can be passed through a gap 320 at the end of the arms 315 and 316. This embodiment is expected to make it easier to suture a relatively small marker while having surgical gloves on or while handling the needle by needle holders.

FIG. 4 is an isometric view of an implantable marker 400 in accordance with another embodiment of the invention. The marker 400 includes an anchor 414 partially embedded into the casing 110. The anchor 414, more specifically, can have an embedded portion 420 at one end of the marker 400. The anchor 414 can be a metal, ceramic, polymeric loop having a polygonal or circular shape. In the illustrated embodiment, the anchor 414 is a metal rod that is punched or bent into a triangular loop. The anchor 414 forms a passageway 426 to receive the suturing needle for surgical implantation or to enable tissue ingrowth for percutaneous implantation. For suturing, however, it is not necessary for the anchor 414 to form a closed-loop with the casing as explained above.

In another embodiment of the invention, illustrated in FIG. 5, a marker 500 can have a first anchor 510 at one end of the casing 110 and a second anchor 520 at the other end of the casing 110. The first and second anchors 510 and 520 can be embedded in or attached to the casing 110 as explained above with respect to FIG. 4.

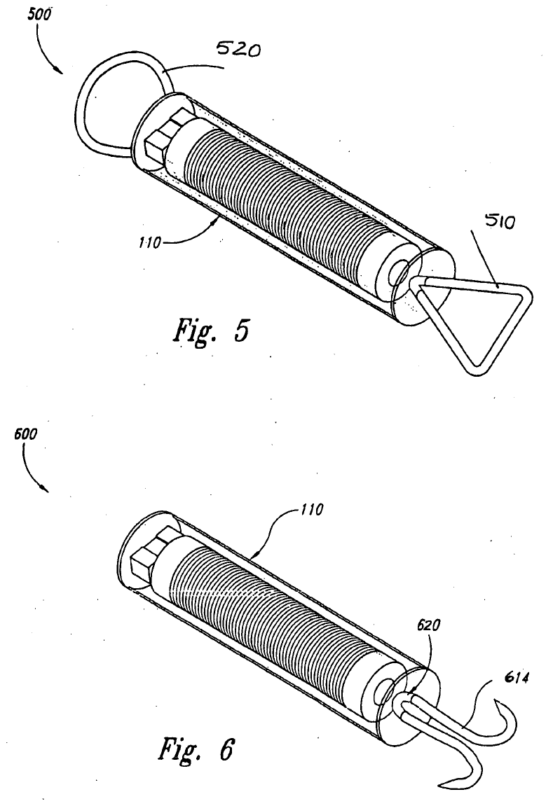

FIG. 6 is an isometric view of the implantable marker 600 having an anchor 614 defined by two adjacent hooks projecting away from the casing 110 in accordance with another embodiment of the invention. The two hooks may be manufactured from the two ends of a single piece of material having a bend 620 to bring the two hooks close together. The bend 620 is embedded in the casing material to attach the anchor 614 to the casing 110. The hooks of the anchor 614, however, may be manufactured from separate pieces of material or there may be more than two hooks at each end of the marker. In another embodiment the anchor may only include a single hook. The anchor 614 is suitable for percutaneous and/or surgical implantation.

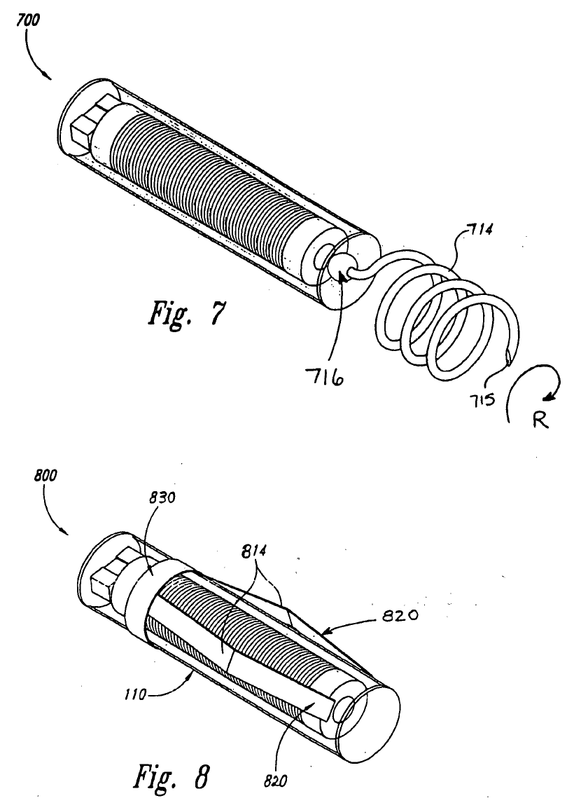

In yet another embodiment of the invention shown in FIG. 7, a marker 700 includes an anchor 714 defined by a helical member having a sharp end. The anchor 714 also includes a sharp end 715 for piercing tissue and a base 716 configured to be embedded in the casing 110. The base 716, for example, can be a small ball formed at one end of the anchor 714. In operation, the marker 700 is rotated as shown by arrow R to attach the marker 700 to the tissue. The marker 700 can be rotated by hand for surgical implantation or by a device similar to a laparoscopic device for percutaneous implantation. In another embodiment, the anchor 714 can be configured as a bone screw secured to the casing 110. The marker 700 can act as the bone screw shaft to allow the marker and anchor 714 to be rotated and embedded in a bone or other rigid material.

FIG. 8 is an isometric view of an implantable marker 800 in accordance with another embodiment of the invention. The marker 800 includes an anchor 814 having spring elements 820 and a ring 830 at one end of the spring elements 820. In an undeployed state, the spring elements 820 extend longitudinally over the surface of the casing 110, and the ring 830 wraps around the circumference of the casing 110. The ring 830 may be adhered to the casing 110. The spring elements 820 can be made from spring steel, “memory” metal alloys, polymeric materials, or other materials that can move outwardly upon deployment. Once deployed, the spring elements 820 move outwardly to project away from the surface of the casing 110. The spring elements 820 can pierce or otherwise press against tissue to hold the marker 800 in place.

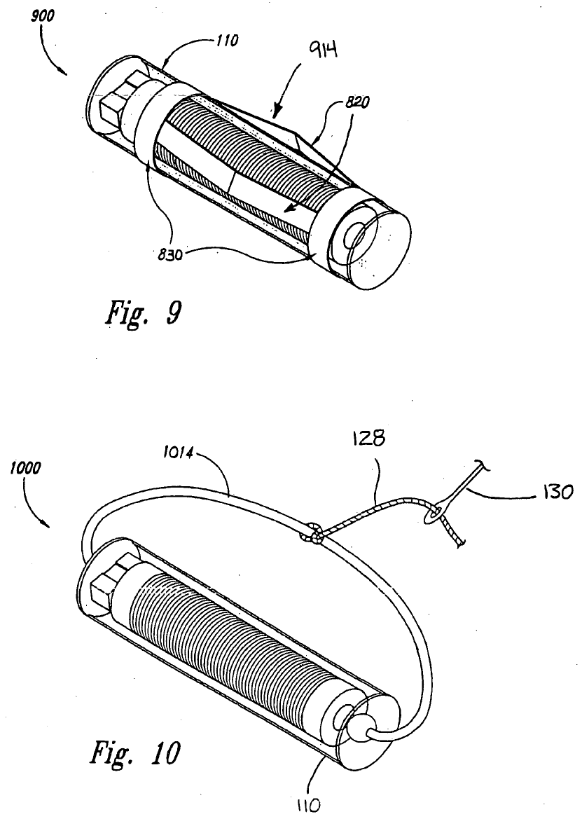

In another embodiment of the invention shown in FIG. 9, a marker 900 has an anchor 914 with the spring elements 820 and a ring 830 at both ends of the spring elements 820. The rings 830 wrap around the circumference of the casing 110 of the marker 900 as depicted above with reference to FIG. 8. To accommodate the movement of the spring elements 820, one of the rings 830 is free to slide over the casing 110 and the other ring 830 can be fixed to the casing 110. Once deployed, the spring elements 820 project from the surface of the casing 110 as explained above.

The markers 800 and 900 are well suited for percutaneous implantation because they can fit within a cannula of an introducer in the undeployed state and automatically expand upon being ejected from the introducer. In another embodiment, the markers 800 and 900 can be surgically implanted by passing a suture line between the spring elements 820 and the casing 110. For example, a needle may be threaded to suture wire and the suture line may be tied to one of the spring elements 820.

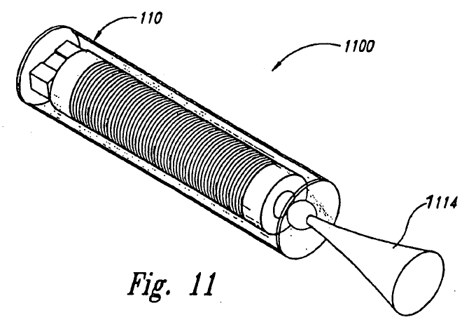

FIG. 10 is an isometric view of an implantable marker 1000 in accordance with yet another embodiment of the invention. The marker 1000 has an anchor 1014 defined by a strap attached to or embedded in both ends of the casing 110. The anchor 1014 is a suture retainer to which suture line can be tied. The anchor 1014 can be flexible or rigid. In the illustrated embodiment, the anchor 1014 has a first ball molded into one end of the casing 110 and a second ball molded into the other end of the casing 110.

FIG. 11 is an isometric view of the implantable marker 1100, which comprises an anchor 1114 defined by a golf-tee shaped member partially embedded in the casing 110. The anchor 1114 can be embedded in the casing at the time of molding the casing 110.

One specific process of using the marker simply involves opening a marker kit with the suture line pre-attached to the marker, and then suturing the marker to the tissue. Depending on the anchor type, the suture line may be passed through at least a hole of the anchor, wrapped around the anchor, or embedded in the casing. If the suture line is embedded directly in the casing 110, the suture line itself defines the anchor.

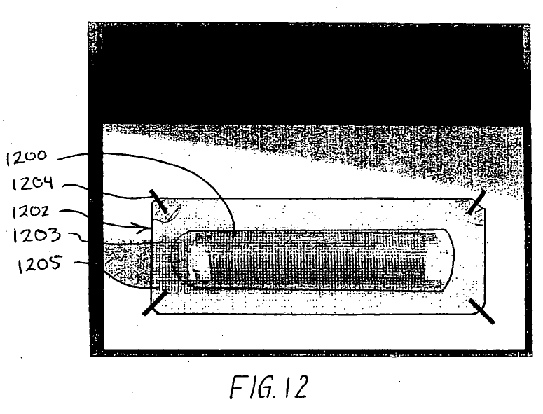

FIG. 12 is an isometric view of a marker 1200 in accordance with another embodiment of the invention. The marker 1200 is secured to a carrier 1202 made of a layer of material 1203 that can be secured in or on a patient using sutures, adhesives, staples, or other fixation means. In the illustrated embodiment, the carrier 1202 is a woven fabric sleeve with the marker 1200 contained in the sleeve. The edge portions 1205 of the carrier 1202 are configured to receive and securely retain sutures 1204 to fix the marker in place at the target location in or on a patient. In one embodiment, portions of the carrier 1202 to which the sutures 1204 attach can be reinforced with material for additional strength.

The material 1203 of the carrier 1202 can be a woven, fabric material having an open weave to allow for in-growth of tissue into the material for additional anchoring of the marker 1200 in or on the patient. In other embodiments, the material 1203 can have a tight weave that substantially blocks in-growth of tissue. The material 1203 can also be a mesh (fabric or non-fabric) with openings of a size suitable for the intended use of the carrier 1202.

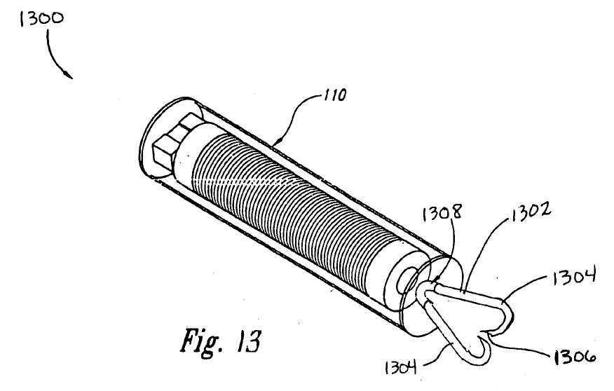

FIG. 13 is an isometric view of an implantable marker 1300 having an anchor 1302 defined by two adjacent hooks 1304 projecting away from the casing 110 in accordance with another embodiment of the invention. Each of the two hooks 1304 have sharp engagement ends 1306 spaced away from the casing 101. In one embodiment, the hooks 1304 are made of clamp members similar to vascular clamps, although other hook devices could be used. The hooks 1304 are oriented so the engagement ends 1306 are generally adjacent to each other when in an anchoring position as shown. In one embodiment, the hooks 1304 can be substantially coplanar so that the hooks define an interior area 1308 between them that allows for in-growth of tissue after the marker 1300 has been implanted. In another embodiment, the hooks 1304 can be oriented in different planes to provide offset engagement ends 1306 for engagement with tissue.

The hooks 1304 can be manufactured from a single piece of material having a bend 1308 to bring the two hooks 1304 close together. In another embodiment, the hooks 1304 can be manufactured from separate pieces of material. There also may be more than two hooks 1304 at one or both ends of the marker 1300. As an example, a marker 1300 in one embodiment can include the hooks 1304 facing each other (FIG. 13) and other hooks facing away from each other (FIG. 6).

In one embodiment, the anchor 1302 can be manufactured of a shape memory material that automatically moves from one shape to another upon application of certain conditions. As an example, the anchor 1302 may be made from Nitinol wire that will return to a memory shape (e.g., the hook shape) at body temperature. In this embodiment, the marker 1300 and anchor 1302 can be introduced into a site through a catheter or other introducer (not shown) while the hooks 1304 are generally straight and extend away from the marker 1300. After the marker 1300 and anchor 1302 are deployed from the catheter, body heat from the patient will cause the Nitonal wire forming the hooks 1304 to automatically return to its memory shape, namely the hook shape.

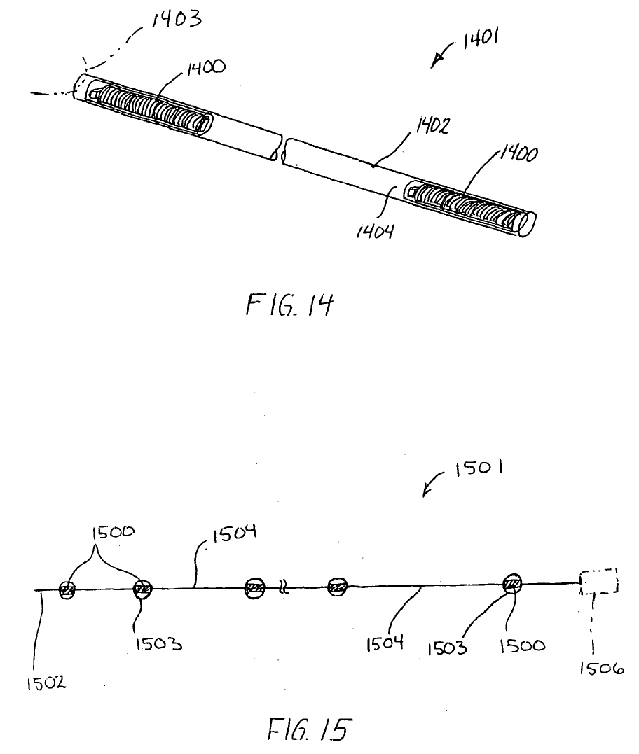

FIG. 14 is an isometric view of a marker anchoring system 1401 that includes multiple markers 1400 in a carrier 1402. The markers 1400 are securely attached to the carrier 1402 to maintain a known axial relationship between the markers 1400. In the illustrated embodiment, the carrier 1402 is a catheter with a lumen 1404 that contains the markers 1400. The markers 1400 are adhered to the walls of the lumen 1404, although other attachment means could be used. The carrier 1402 can be substantially rigid so that the axial and lateral spacing between the markers 1400 remains fixed. In another embodiment, the carrier 1402 may be a flexible member that can bend so that the markers 1400 do not have to be positioned in a straight line.

The carrier 1402 containing the markers 1400 can be implanted in a patient surgically or percutaneously through a catheter or other introducer. The carrier 1402 and markers 1400 are configured to be easily removed as a unit from the patient. As an example, the markers 1400 can be removed from a patient simply by pulling axially on an end of the carrier 1402 and removing it from the patient. In one embodiment, the carrier 1402 is configured to be fixed in place in a patient with sutures 1403 or other anchoring device.

FIG. 15 is a side elevation view of a marker anchoring system 1501 having a carrier 1502 with a plurality of markers 1500 in accordance with another embodiment of the invention. The carrier 1502 includes markers 1500 embedded in a plurality of beads 1503 that are interconnected by connecting members 1504. In the illustrated embodiment, the beads 1503 are made of a dielectric material that encases the markers 1500. The beads 1503 can be spherical, spheroidal, ellipsoidal, or another geometric shape. The connecting members 1504 can be thread, wire, line, or other material that securely interconnects all of the beads 1503 together. The connecting members 1504 can be axially rigid or can be flexible so that the beads 1503 and markers 1500 can be implanted surgically or percutaneously in a selected orientation relative to a target while maintaining the known spatial relationship between the markers 1500.

The marker anchoring system 1501 of the illustrated embodiment is configured to be implanted in soft tissue, such as a breast, from which the markers 1500 typically would be removed after completion of a procedure or series of procedures. The marker anchoring system 1501 can be removed by grasping one end of the carrier 1502 and pulling axially. Accordingly, the connecting members 1504, beads 1502, and markers 1500 are removed as a unit and in one motion. In one embodiment, a tab or other grip portion 1506 (shown in phantom lines) may be provided on an end of the fixation device 1502. The grip portion 1506 is configured so that a surgeon can securely grasp the grip portion and pull axially on the carrier 1502.

FIG. 16 is a marker anchoring system 1600 with a plurality of the markers 1500 in accordance with another embodiment of the invention. The marker anchoring system 1600 includes the carrier 1502 with the beads 1503, the markers 1500 and the connecting members 1504 discussed above. The marker anchoring system 1600 also includes an external fixation member 1602 on or adjacent to an end segment 1608 of the connecting members 1504. The marker anchoring system 1600 can be implanted surgically or percutaneously into target tissue 1606, but at least a portion of the end segment 1608 and the fixation device 1602 remain exterior of the target tissue.

As an example, the beads 1503, markers 1500, and connecting members 1504 can be implanted in breast tissue, and a portion of the end segment 1608 and the fixation device 1602 remain adjacent to the surface tissue 1610, such as the skin of the breast. The fixation device 1602 is configured to be securely fixed to the surface tissue 1610 with sutures, adhesive, staples, or other fixation mechanisms. The market anchoring system 1600 can also be used in internal cavities that allow access to the fixation device 1602. As an example, the marker anchoring system 1600 could be used in the cervix area for treatment of cervical cancer. The fixation device 1602 can be released from the surface tissue 1610 and the marker anchoring system 1600 removed as a unit from the target tissue 1606 by pulling axially on the end segment 1608 and/or the fixation device away from the target tissue.

In another embodiment, the marker anchoring system 1600 has a fixation device 1602 that can be secured subcutaneously as the markers 1500 are implanted. The subcutaneous fixation device 1602 is palpable so that the surgeon can easily determine through touch or visual inspection where the marker anchoring system 1600 is located. In one embodiment, the fixation device 1602 can be positioned subcutaneously, but a portion of the end segment 1608 could extend through the surface tissue 1610 to provide a visible exterior marker indicating the location of the fixation device 1602. A small incision could then be made in the surface tissue 1610 to access the fixation device 1602 for removal of the entire marker anchoring system 1600.

In one embodiment, the fixation device 1602 can be a collapsible fixation device that could be passed through a catheter in a collapsed position as the marker anchoring system 1600 is being implanted into the target tissue 1606. The fixation device 1602 automatically moves to an expanded position upon exiting the catheter. In one embodiment, the fixation device 1602 can be made of Nitonal or other shape memory materials. In another embodiment, the fixation device 1602 can be separate from the end segment 1608, so the fixation device does not pass through the catheter. The fixation device 1602 can be attached to the end segment 1608 and to the surface tissue 1610 after the markers 1500 are implanted.

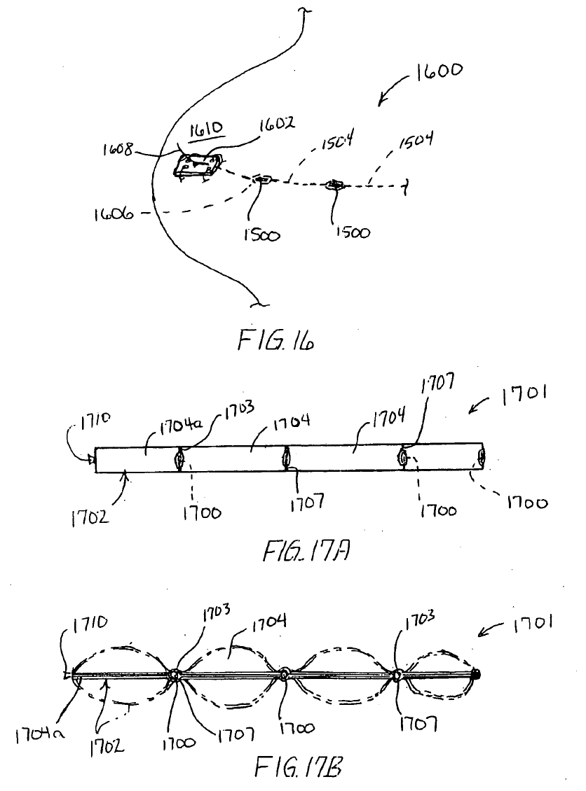

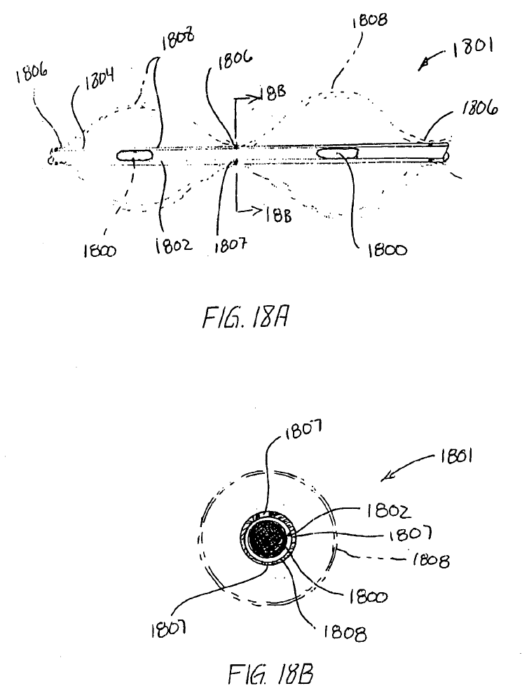

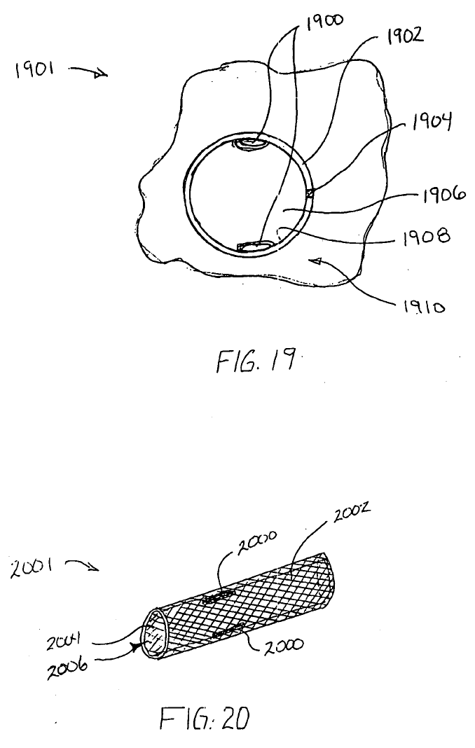

FIG. 17A is a top plan view of a marker anchoring system 1701 with a plurality of markers 1700 attached to a carrier 1702 in accordance with another embodiment. FIG. 17B is a side elevation view of the marker anchoring system 1701 of FIG. 17A. The carrier 1702 includes a plurality of inflatable segments 1704 interconnecting the plurality of markers 1700. In one embodiment, the markers 1700 are embedded in non-inflatable portions 1703 of the carrier 1702 adjacent to the inflatable segments 1704. The inflatable segments 1704 are moveable between a substantially flat configuration (shown in solid lines) and an inflated configuration (shown in phantom lines in FIG. 17B).

In one embodiment, the carrier 1702 can be made from an elastic and inflatable material that is used in conventional balloon angioplasty. The carrier 1702 can also be constructed of a radio-opaque material that allows the fixation device to be identifiable with conventional imaging techniques used in treatment planning or to identification of locations needing enhanced imaging.

The marker anchoring system 1701 is configured so that the segments 1704 can be filled with air, saline, or other suitable inflating material to expand the segments to the inflated configuration. In one embodiment, the inflation material is introduced through an inflation/deflation valve 1710 in fluid communication with a first inflatable segment 1704a. In one embodiment, passageways 1707 extend through the non-inflatable portions 1703 so that the inflating material can pass between the inflatable segments 1704 during inflation or deflation of the segments. Accordingly, all of the inflatable segments 1704 can be inflated by directing the inflating material under a suitable pressure through the valve 1710 and into the first inflation segment 1704a. The inflation material will then migrate through the passageways 1707 in the non-inflatable portions 1703 and into all of the inflatable segments 1704. The inflation material will cause the inflatable segments 1704 to move from the flat configuration to the inflated configuration.

In use, the marker anchoring system 1701 can be implanted surgically or percutaneously when the inflatable segments 1704 are in the flat configuration so that the markers 1700 are positioned in a known relationship to each other. The inflation material can then be introduced into the carrier 1702 to expand the inflatable segments 1704 in a manner known in the art of balloon catheters. The segments 1704 in the inflated configuration anchor the carrier 1702 in a fixed position within a selected body cavity or passageway, such as a lung, vessel, or other structure. The segments 1704 can be deflated and moved substantially back to the flat configuration by removing the inflation material through the inflation/deflation valve 1710. After the segments 1704 have been deflated, the marker anchoring system 1701 can be removed as a unit by pulling axially on one end of the carrier 1702 as discussed above.

FIG. 18A is a side elevation view of a marker anchoring system 1801 in accordance with another embodiment. The marker anchoring system 1801 includes an expandable sleeve 1804 covering a carrier 1802 that contains a plurality of markers 1800. In the illustrated embodiment, the carrier 1802 is a catheter, although other carriers could be used in other embodiments. The markers 1800 are fixed in the carrier 1802 in a known relationship relative to each other. The expandable sleeve 1804 is fixed to the carrier 1802 at spaced apart connection portions 1806. In the illustrated embodiments, the markers 1800 are positioned in the carrier 1802 between the connection portions, although the markers can be in other locations within the carrier. The sleeve 1804 is not fixed to the carrier 1802 along expandable segments 1808 extending between the connection portions 1806. The expandable segments 1808 are configured to be expanded with air, saline, or other inflation material from a flat configuration (shown in FIG. 18A in solid lines) to an inflated configuration (shown in phantom lines in FIGS. 18A and 18B).

The sleeve 1804 is configured so the inflation material can pass into the lumen of the expandable sleeve 1804 and around the exterior surface of the carrier 1802. The inflation material enters the sleeve 1804 through an inflation/deflation valve (not shown) and flows through passageways 1807 in the connection portions 1806 and into the expandable segments 1808 to inflate the expandable segments 1808 around the carrier 1802. Each expandable segment 1808, when in the inflated configuration, anchors the marker anchoring system 1801 in a fixed position within a selected body cavity. The expandable segments 1804 can be deflated to the flat configuration by removing the inflation material from the sleeve 1804 through the inflation/deflation valve. The marker anchoring system 1801 can then be removed as a unit from the body cavity by pulling axially on the carrier 1802 or on the sleeve 1804.

FIG. 19 is a side elevation view of a marker anchoring system 1901 having a plurality of markers 1900 connected to an inflatable carrier 1902 in accordance with another embodiment. The inflatable carrier 1902 is a balloon member constructed of an expandable material used in conventional balloon catheter. The inflatable carrier 1902 can also include a substantially radio-opaque material that allows the inflatable carrier to be imaged with conventional CT scans and X-rays. The inflatable carrier 1902 has an inflation/deflation valve 1904 that allows air, saline, or other inflatable material to be added to and removed from the interior area 1906 of the inflatable carrier as known in the art of balloon catheters. A plurality of markers 1900 are secured to the inner wall 1908 of the inflatable carrier 1902 so that the markers will be immediately adjacent to the inner wall of a body cavity in the patient when the inflatable member is fully inflated.

In the illustrated embodiment, the inflatable carrier 1902 is configured to fit within a selected body cavity 1910, such as a bladder, lung, or other cavity. In one embodiment, an anchor (not shown) can be coupled to the inflatable carrier 1902 for additional anchoring of the inflatable carrier to a portion of the cavity 1910. As an example, the inflatable carrier 1902 can be provided with hooks, clips, helical members, or other anchoring mechanisms discussed above. The inflatable carrier 1902, when inflated, securely holds the marker anchoring system 1901 in place in the cavity 1910 until the inflatable carrier is deflated. After the inflatable carrier 1902 is deflated by removing the inflating material through the valve 1904, the inflatable carrier and the markers 1900 can be removed from the cavity 1910 as a unit.

FIG. 20 is an isometric view of a marker anchoring system 2001 with a plurality of markers 2000 on a carrier 2002 in accordance with another embodiment. The carrier 2002 is a stent formed of a radially expanding member configured to be inserted into lumen in the body, such as a vein, artery or other lumen. The carrier 2002 can be a suitable stent known in the art of cardiac and vascular treatment. The carrier 2002 has sidewalls 2004 that define a lumen 2006, and the markers 2000 are fixed to the sidewalls within the lumen. The markers 2000 are retained in a known relationship relative to each other. In one embodiment, the markers 2000 are embedded in the sidewalls 2004 so that the markers do not significantly decrease the cross-sectional area of the lumen 2006. The markers 2000 can be embedded in a dielectric material connected or embedded in the sidewall 2004 so that medicament can be provided in or on the carrier 2002, such as a stent, without actually contacting the marker.

From the foregoing, it will be appreciated that specific embodiments of the invention have been described herein for purposes of illustration, but that various modifications may be made without deviating from the spirit and scope of the invention. For example, the anchors can be composed of more than one material, or the anchors of the various embodiments can be interchanged or combined with each other. Additionally, some surgically implantable markers are also well adapted for percutaneous implantation. In cases of percutaneous implantation the anchors will lodge in the surrounding tissue instead of being sutured to the tissue. In some cases, depending on the geometry of the anchor, the surrounding tissue may grow into holes or other interstitial spaces of the anchor. The cylindrically shaped markers with anchors only at one end, such as in FIG. 6, are suited for percutaneous as well as surgical implantation. Furthermore, the anchors can further comprise drug eluting surfaces that contain drugs to promote tissue growth, provide antibodies, etc. Anchoring capacity can also be enhanced by adding adhesive material to the anchor and/or the casing. Accordingly, the invention is not limited except as by the claims.

What is claimed is:

1. A wireless marker for implantation into a human body, comprising:
a casing;
a signal element at least partially encased in the casing, the signal element being configured to wirelessly transmit a location signal in response to a wirelessly transmitted excitation energy; and
an anchor projecting from the casing, the anchor having a suture retainer configured to receive a suture line.

2. The wireless marker of claim 1 wherein the signal element comprises a magnetic permeable core, a coil wrapped around the core, and a capacitor electrically connected to the coil.

3. The wireless marker of claim 1 wherein a suture line is attached to the suture retainer.

4. The wireless marker of claim 1 wherein the anchor further comprises adhesive material that binds and/or adheres to tissue.

5. The wireless marker of claim 1 wherein the anchor further comprises a drug.

6. The wireless marker of claim 1 wherein the anchor comprises a fin projecting from the casing and the suture retainer is at the fin.

7. The wireless marker of claim 1 wherein the anchor comprises a fin projecting from the casing and the suture retainer comprises a hole through the fin.

8. The wireless marker of claim 1 wherein the anchor comprises a first anchor member projecting from one portion of the casing, a second anchor member projecting from another portion of the casing, and the suture retainer comprises a hole through one of the first or second anchor members.

9. The wireless marker of claim 1 wherein the anchor comprises a flange projecting from the casing, wherein the flange is about as thick as the casing.

10. The wireless marker of claim 1 wherein the anchor comprises a stem projecting from the casing, a first arm projecting from the stem in one direction, and a second arm projecting from the stem in another direction.

11. The wireless marker of claim 1 wherein the anchor comprises a flange having a stem, a first arm projecting in one direction from the stem, and a second arm projecting in a different direction from the stem.

12. The wireless marker of claim 1 wherein the anchor comprises a rigid loop having a base embedded into the casing.

13. The wireless marker of claim 1 wherein the anchor comprises a first loop having a first base embedded into one portion of the casing and a second loop having a second base embedded into a second portion of the casing.

14. The wireless marker of claim 1 wherein the anchor comprises a double hook having a medial portion embedded into the casing.

15. The wireless marker of claim 1 wherein the anchor comprises a helical member having a first end configured to pierce tissue and a second end having a base embedded in the casing.

16. The wireless marker of claim 1 wherein the anchor comprises a ring secured to the casing and a plurality of spring elements attached to the ring, wherein the spring elements move from an undeployed position in which the spring elements are proximate to the casing to a deployed position in which the spring elements are spaced apart from the casing by a greater distance.

17. The wireless marker of claim 1 wherein the anchor comprises a strap having a first end attached to one portion of the anchor and a second end attached to a different portion of the anchor.

18. The wireless marker of claim 17 wherein a suture line is attached to the strap.

19. A wireless marker for implantation into a human body, comprising:
a biocompatible casing;
a transponder at least partially encased in the casing, the transponder having a core, a coil wrapped around the core, and a capacitor connected to the coil; and
an anchor carried by the casing, the anchor includes a hole configured to receive a suture line.

20. The wireless marker of claim 19 wherein a suture line is pre-attached to the anchor.

21. The wireless marker of claim 19 wherein the anchor further comprises adhesive material that binds and/or adheres to tissue.

22. The wireless marker of claim 19 wherein the anchor further carries a tissue growth enhancing drug.

23. A wireless marker, comprising:
a biocompatible casing configured for implantation into a human body;
a signal element at least partially encased in the casing, the signal element having a resonating circuit configured to wirelessly transmit a location signal in response to a wirelessly transmitted excitation energy;
an anchor configured to hold the wireless marker at a reference location in the human body relative to a target location, wherein the anchor projects out of the casing; and
a suture line pre-attached to the anchor.

24. A wireless marker for implantation into a human body, comprising:
a casing;
a signal element at least partially encased in the casing, the signal element having a transponder configured to wirelessly-transmit a location signal in response to a wirelessly transmitted excitation energy; and
an anchor at least partly embedded in the casing and configured to contact an anatomical medium for holding the wireless marker at an implant site in the human body.

25. The wireless marker of claim 24 wherein the anchor includes a base embedded in the casing material.

26. The wireless marker of claim 24 wherein the signal element comprises a resonating circuit including a ferrite core, a coil wrapped around the core, and a capacitor connected to the coil.

27. The wireless marker of claim 24 wherein suturing means are attached to the anchor.

28. The wireless marker of claim 24 wherein the anchor member further comprises a suture retainer configured to receive a suture line.

29. The wireless marker of claim 24 wherein the anchor member is golf-tee-like.

30. The wireless marker of claim 24 wherein the anchor comprises a medial section embedded in the casing, a first hook extending from the medial section, and a second hook proximate the first hook.

31. The wireless marker of claim 24 wherein the anchor comprises a spring element extending longitudinally along the casing and a ring attached to one end of the spring element, wherein the ring is attached to the casing.

32. The wireless marker of claim 24 wherein the anchor comprises a closed-loop having a triangular shape, and wherein a corner of the triangular loop is embedded in the casing.

33. A wireless marker, comprising:
a biocompatible casing configured for implantation into a human body;
a signal element at least partially encased in the casing, the signal element having a resonating circuit configured to wirelessly transmit a location signal in response to a wirelessly transmitted excitation energy;
a carrier connected to the casing; and
an anchor connected to the carrier and configured to hold the carrier and wireless marker at a reference location on the human body relative to a target location.

34. The wireless marker of claim 33 wherein the carrier is a fabric sleeve that contains the casing.

35. The wireless marker of claim 33 wherein the carrier is a mesh layer.

36. The wireless marker of claim 33 wherein the anchor is a suture that attached to a reinforced portion of the carrier.

37. A marker anchoring system for implantation of wireless markers into or onto a human body, comprising:
a carrier anchorable to the human body; and
a plurality of wireless markers coupled to the carrier at a known relationship relative to each other, the wireless markers having a casing attached to the carrier and a signal element at least partially encased in the casing, the signal element being configured to wirelessly transmit a location signal in response to a wirelessly transmitted excitation energy.

38. The marker anchoring system of claim 37 wherein the carrier is a catheter, and the markers are positioned within the catheter.

39. The marker anchoring system of claim 37 wherein the carrier includes a plurality of beads encasing the markers and connection members interconnecting the plurality of beads.

40. The marker anchoring system of claim 39 wherein the carrier includes a gripping portion attached to at least one of the connection members.

41. The marker anchoring system of claim 39 wherein the carrier includes a fixation device connected to at least one of the connection members.

42. The marker anchoring system of claim 37 wherein the carrier has a plurality of inflatable segments movable between an inflated configuration and a deflated configuration.

43. The marker anchoring system of claim 37 wherein the carrier includes a catheter with a lumen, the plurality of markers being coupled to the catheter adjacent to the lumen, and an outer member connected to the catheter, the outer member having inflatable segments movable between an inflated configuration and a deflated configuration.

44. The marker anchoring system of claim 37 wherein the carrier is an expandable balloon structure with sidewalls that define an interior area, the plurality of markers are coupled to the sidewalls adjacent to the interior area.

45. The marker anchoring system of claim 37, wherein the carrier is a stent.

46. A method of making an anchorable wireless marker with an embedded anchor member, comprising:
placing a portion of an anchor within a casing mold; and
casting the casing material into the mold to embed a portion of the anchor in the casing material.
